(19) United States
(12) Patent Application Publication
(10) Pub. No.: US 2011/0294828 A1
(43) Pub. Date: Dec. 1, 2011

(54) PROCESS FOR PREPARING A POLYMORPH OF THE CHOLINE SALT OF A PYRIMIDIN-5-YL ACETIC ACID DERIVATIVE

(75) Inventors: Dhileepkumar Krishnamurthy, Brookfield, CT (US); Juan Manuel Rodriguez Dehli, Mainz (DE); Michael Schul, Lahnstein (DE); Xiao-Jun Wang, Danbury, CT (US); Bing-Shiou Yang, Southbury, CT (US)

(73) Assignee: BOEHRINGER INGELHEIM INTERNATIONAL GMBH, Ingelheim am Rhein (DE)

(21) Appl. No.: 12/950,099

(22) Filed: Nov. 19, 2010

Related U.S. Application Data
(60) Provisional application No. 61/263,871, filed on Nov. 24, 2009.

(51) Int. Cl. A61K 31/505; A61P 11/00; A61P 31/12; A61P 11/06; A61P 31/00; A61P 31/04; C07D 239/48; A61P 11/08

(52) U.S. Cl. 514/256; 544/329

(57) ABSTRACT

Provided is a process for preparing a choline salt of [4,6-bis(dimethylamino)-2-(4-{[4-(trifluoromethyl)benzoyl]amino}benzyl)pyrimidin-5-yl]acetic acid. The process of the invention is useful for preparing the salt in purer forms of the salt. Also disclosed is a more pure form of the of choline salt of [4,6-bis(dimethylamino)-2-(4-{[4-(trifluoromethyl)benzoyl]amino}benzyl)pyrimidin-5-yl]acetic acid.

### FIELD OF THE INVENTION

This invention relates to a process for preparing a choline salt of [4,6-bis(dimethylamino)-2-(4-{[4-(trifluoromethyl)benzoyl]amino}benzyl)pyrimidin-5-yl]acetic acid. The process of the invention is useful for preparing purer forms of the salt.

### BACKGROUND OF THE INVENTION

CRTH2 is a G protein-coupled chemoattractant receptor expressed on Th2 cells, eosinophils, and basophils (Nagata et al., J. Immunol. 1999, 162, 1278-1286; Hirai et al., J. Exp. Med. 2001, 193, 255-261). Prostaglandin D2 (PGD2), the major inflammatory mediator produced from mast cells, is a natural ligand for CRTH2. Recently, it has been shown that the activation of CRTH2 by PGD2 induces the migration and activation of Th2 cells and eosinophils, suggesting that CRTH2 may play a pro-inflammatory role in allergic diseases (Hirai et al., J. Exp. Med. 2001, 193, 255-261; Gervais et al., J. Allergy Clin. Immunol. 2001, 108, 982-988). It has also been shown that, in atopic dermatitis patients, there is an increase in circulating T cells expressing CRTH2, which correlates with the severity of the disease (Cosmi et al., Eur. J. Immunol. 2000, 30, 2972-2979; Iwazaki et al., J. Investigative Dermatology 2002, 119, 609-616). The role of PGD2 in the initiation and maintenance of allergic inflammation has further been demonstrated in mouse models of asthma by showing that overproduction of PGD2 in vivo by PGD2 synthase exacerbates airway inflammation (Fujitani et al., J. Immunol. 2002, 168, 443-449). Therefore, CRTH2 antagonists are potentially useful for the treatment of CRTH2-mediated disorders or diseases, such as allergic rhinitis, allergic asthma, bronchoconstriction, atopic dermatitis, or systemic inflammatory disorders.

International Publication No. WO2008/15678 discloses the free-acid form of [4,6-bis(dimethylamino)-2-(4-{[4-(trifluoromethyl)benzoyl]amino}benzyl)pyrimidin-5-yl]acetic acid, which has the formula (I),

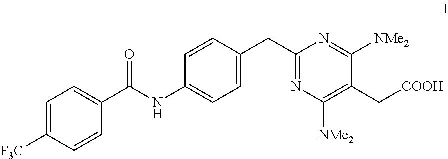

and reports that the compound is useful as a CRTH2 antagonist.

International Publication No. WO2008/156780 discloses two crystalline polymorphs of the free-acid form of the compound of formula (I).

International Publication No. WO2008/156781 discloses amine salts of the compound of formula (I) including a crystalline choline salt. However, WO2008/156781 does not describe the yield of choline salt produced by disclosed process or its purity.

Disclosed herein is an improved process for preparing the crystalline choline salt of the compound of formula (I), which provides said choline salt in high yield and high purity.

### SUMMARY OF THE INVENTION

In its broadest embodiment, the invention relates to a process for preparing a crystalline form of the choline salt of the compound of formula (I), the process comprising:
(a) forming a first admixture of a choline salt of the compound of formula (I) in a solvent comprising isopropanol and water;
(b) contacting the first admixture of Step (a) with an anti-solvent to provide a second admixture; and
(c) allowing the choline salt of the compound of formula (I) to crystallize from said second admixture of Step (b) to provide the crystalline form of the compound of formula (I).

For convenience, the process described immediately above is referred to herein as the “process of the invention” or “processes of the present invention.”

The choline salt produced by the process of the invention provide an X-ray powder diffraction pattern comprising 2θ angles of about 6.6, 15.2, 16.1, 18.6, 19.5, 20.0, 21.6, 26.5°, which is substantially similar to the X-ray powder diffraction pattern described in WO2008/156781 for the choline salt of the compound of formula (I).

In another embodiment, the invention relates to a crystalline choline salt of the compound of formula (I) (“the choline salt of the invention”), wherein said crystalline choline salt of the compound of formula (I) contains less than about 0.30 wt. % of 2-(4-(dimethylamino)-6-hydroxy-2-4-(trifluoromethyl)benzamido)pyrimidine-5-yl)acetic acid (Compound A) and N-(4-(5-(cyanomethyl)-4,6-bis(dimethylamino)pyrimidin-2-yl)methyl)phenyl)-4-(trifluoromethyl)benzamide (Compound B):

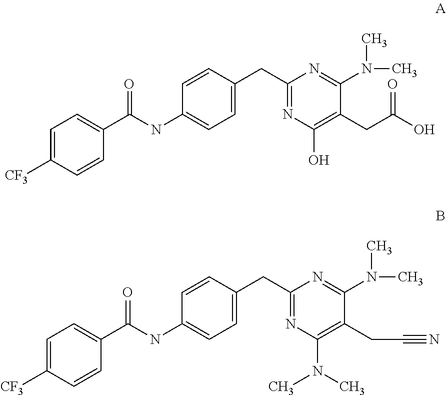

based on the total weight of Compound A, Compound B, choline, and the compound of formula (I).

In yet another embodiment, the invention relates to a pharmaceutical composition comprising a pharmaceutically effective amount of the choline salt of the invention, at least one of a pharmaceutically acceptable carrier or excipient and, optionally, one or more further active compounds (“the pharmaceutical composition of the invention”). In another embodiment, the invention relates to a method of treating or preventing one or more symptoms of a CRTH2-mediated disease or disorder comprising administering to a patient a therapeutically effective amount of the choline salt of the invention.

### BRIEF DESCRIPTION OF THE DRAWINGS

FIG. 1 depicts an X-ray powder diffraction pattern of the choline salt of the compound of formula (I) produced by the process of the invention.

FIG. 2 depicts a differential scanning calorimetric (DSC) and thermal gravimetric analysis (TGA) thermograms of the choline salt of the compound of formula (I) produced by the process of the invention.

FIG. 3 depicts a dynamic vapor sorption (DVS) isotherm plot of the choline salt of the compound of formula (I) produced by the process of the invention.

### DETAILED DESCRIPTION OF THE INVENTIONS

As noted above, the subject invention relates to a process for preparing a crystalline form of the choline salt of the compound of formula (I), the process comprising:
(a) forming a first admixture of a choline salt of the compound of formula (I) in a solvent comprising isopropanol and water (“the admixing step”);
(b) contacting the first admixture of Step (a) with anti-solvent (“the anti-solvent addition step”) to provide a second admixture; and
(c) allowing the choline salt of the compound of formula (I) to crystallize from said second admixture of Step (b) to provide the crystalline form of the compound of formula (I) (“the crystallization step”).

Applicants have found that process of the present invention provides the choline salt of the compound of formula in higher yield and higher purity than does the process described in WO 2008/156781. The process of the present invention is also more amenable to large-scale production than is the process described in WO 2008/156781, because it provides better design of crystallization and choices of solvents to inhibit the hydrolysis of the compound In one embodiment, the process of the invention also uses milled seeds that can directly produce the final product with the desired particle size distribution, thereby avoiding the need of milling the product.

The choline salt of the compound of formula (I) produced by the process of the invention is characterized by an X-ray powder diffraction pattern comprising 2θ angles and d-spacing values as shown in Table 1 (see below). The values reported in Table 1 for the choline salt of the compound of formula (I) are substantially similar to those reported in WO 2008/156781, indicating that the processes produce substantially similar polymorphs.

The choline salt of the compound of formula (I) used in the admixing step can be in the form of a pre-formed solid such as crystalline or amorphous solid; a solvate (e.g., hydrate) or ansolvate (e.g., anhydrate); or any combination of the foregoing.

Alternatively, the choline salt of the compound of formula (I) used in the admixing step can be in the form of a liquid, e.g., solution or slurry comprising (1) the choline salt of the compound of formula (1) and (2) a solvent comprising isopropanol, water, or a combination thereof.

In another alternative, the choline salt of the compound formula (I) used in the admixing step above may be generated or formed in-situ by allowing a free-acid form of the compound of formula (I) and choline hydroxide to react in isopropanol and water to form the choline salt of the compound formula (I) (“the salt-forming step”).

As used herein, the term “free-acid” as it relates to the compound of formula (I) refers to non-salt forms of the compound of formula (I).

For the in-situ salt formation, the molar ratio of the free-acid form of the compound of formula (I) to choline hydroxide used for in-situ salt-formation can vary from about 3:1 to 1:3; from about 2.1 to about 1:2; or about 1:1. The resulting crystals of the choline salt of the compound of formula (I) have a molar ratio of the free-acid form of the compound of formula (I) to choline of about 1:1. The form of the compound of formula (I) used for in-situ salt-formation can be a solvate or hydrate of the free-acid form of the compound of formula (I), and can be amorphous or crystalline, e.g., the Form I described in WO2008156780. Alternatively, the free-acid compound of formula (I) used in the salt-forming step can be an amorphous anhydrate and/or ansolvate.

Thus, in another embodiment, the invention relates to a process of making a choline salt of the compound of formula (I) comprising:
(a) combining the free-acid form of the compound of formula (I) with choline hydroxide in the presence of isopropanol and water to provide a first admixture comprising the choline salt of the compound of formula (I) (“the admixing step”);
(b) contacting the first admixture of Step (a) with an anti-solvent (“the anti-solvent addition step”) to provide a second admixture; and
(c) allowing the choline salt of the compound of formula (I) to crystallize from said second admixture of Step (c) to provide the crystalline form of the compound of formula (I) (“the crystallization step”).

The admixing step in the embodiments described above is carried out for a time and at a temperature sufficient to allow at least a majority of the choline salt of the compound of formula (I) to dissolve. Thus, in one embodiment, at least a majority of the choline salt of the compound of formula (I) is dissolved in the admixing step; and in another embodiment, essentially all of the choline salt of the compound of formula (I) is dissolved in the admixing step.

A suitable temperature for the admixing step is from about 25° C. to about the refluxing temperature of the solvent; in another embodiment, from about 25° C. to about 80° C.; in another embodiment, from about 25° C. to about 60° C.; in another embodiment, from about 40° C. to about 65° C.; and in another embodiment, about 60° C. to 65° C. A suitable time for the admixing step is typically from about 15 minutes to about 24 hours; or from about 15 minutes to about 5 hours; or from about 15 minutes to about 2 hours. It will be understood that admixing step may include one or more temperature ramps including plateaus where the temperature may be held constant for a period of time.

The amount of isopropanol and water used in the admixing step will vary depending upon the admixing temperature and the amount of water present in the solvent system. Typically, the total amount of isopropanol and water used in the admixing step is that amount necessary to dissolve substantially all of the choline salt of the compound of formula (I) in the admixture at the admixture temperature. In one embodiment, the total amount of isopropanol/water solvent system used in the admixing step can be from about 25 wt. % to about 95 wt. %; or from about 60 wt. % to about 65 wt. % based on the total weight of isopropanol, water, compound of formula (I) and choline hydroxide. The amount of water present in the isopropanol/water solvent system solvent system can be from about 1 wt. % to about 50 wt. %; or from about 5 wt. % to about 25 wt. %; or about 23 wt. % based on the total weight of isopropanol and water.

In one embodiment, the admixing step is carried out with a solvent comprising isopropanol and water.

In another embodiment, the admixing step is carried out with a solvent consisting essentially of isopropanol and water.

In yet another embodiment, the admixing step is carried out with a solvent consisting of isopropanol and water.

As discussed above, the choline salt can also be generated in-situ during the admixing step (the salt-forming step described above). The salt-forming step, when used, is carried out for a time and at a temperature sufficient to allow at least a majority of the choline hydroxide and free-acid form of the compound of formula (I) to react to form the choline salt. Typically, the salt-forming step is carried out at a temperature from about 25° C. to about the refluxing temperature of the solvent system; in another embodiment, from about 25° C. to about 40° C.; in another embodiment, from about 40° C. to about 65° C.; from about 60° C. to about 70° C.; and in another embodiment, from about 60° C. to about 65° C. A suitable time for the salt-forming step, when used, is typically from about 15 minutes to about 24 hours; or from about 15 minutes to about 5 hours; or from about 15 minutes to about 2 hours It will be understood that salt-forming step may include one or more temperature ramps including plateaus where the temperature may be held constant for a period of time.

The order of addition of the free-acid form of the compound of formula (I) and the choline hydroxide in the salt-forming step is not critical. Typically, a solution of choline hydroxide in water is added to an admixture comprising the free-acid form of the compound of formula (I) and isopropanol. The resultant admixture comprising the compound of formula (I), choline hydroxide and isopropanol and water is then processed in the same manner as described above in the admixing step.

The process of the present invention further comprises an anti-solvent addition step. Non-limiting examples of anti-solvents useful in the process of the invention include acetone, isopropanol and heptane. In a one embodiment, the anti-solvent used in the anti-solvent addition step comprises acetone. In a preferred embodiment, the solvent used in the admixing step consists essentially of isopropanol and water, and the anti-solvent used in the anti-solvent addition step consists essentially of acetone. In another embodiment, the anti-solvent used in the anti-solvent addition step consists of acetone.

The amount of anti-solvent used in the anti-solvent addition step can vary depending upon the temperature of the admixture and the specific anti-solvent used. In general, the anti-solvent is used in an amount sufficient to precipitate (crystallize) at least a majority of the choline salt of the compound of formula (I) formed in the second admixture. The amount of anti-solvent used, in one embodiment, is from 25 wt. % to about 95 wt. %; or from about is from 80 wt. % to about 85 wt. % based on the total amount of isopropanol and water and acetone used in the admixing step. The anti-solvent addition step in the embodiments described above is carried out for a time and at a temperature sufficient to allow at least a majority of the choline salt of the compound of formula (I) precipitate (crystallize) from the second admixture. A suitable time for the anti-solvent addition step is from about 0.25 hours to about 10 hours; or from about 0.5 hours to about 10 hours; or from about 1 hour to about 4 hours.

A suitable temperature for the anti-solvent addition step is, in one embodiment, from about −20° C. to about the refluxing temperature of the resultant solvent system; in another embodiment, from about −10° C. to about 40° C.; and in another embodiment, from about 0° C. to about 40° C. In one embodiment, the anti-solvent addition step is carried out simultaneously as the temperature of the admixture is decreased. The anti-solvent addition step can, in one embodiment, begin when the temperature of the admixture is from 20° C. to about 40° and be completed when the temperature of the admixture is from about −10° C. to about 10° C. In another embodiment, the anti-solvent addition step can begin when the temperature of the admixture is about 40° and is completed when the temperature of the admixture is about 0° C.

The crystallization step in the embodiments described above is carried out for a time and at a temperature sufficient to allow at least a majority of choline salt of the compound of formula (I) to crystallize or precipitate from the second admixture. A suitable temperature for the crystallization step is from about −20° C. to about 40° C.; in another embodiment, from about −10° C. to about 30° C.; and in another embodiment, about 0° C. A suitable time for the crystallization step is typically from about 1 hour to about 72 hours; or from about 1 hour to about 48 hours; or from about 2 hours to about 24 hours. It will be understood that crystallization step may include one or more temperature ramps including plateaus where the temperature may be held constant for a period of time.

In one embodiment, the process of the invention further comprises the step of seeding the first admixture of Step (a) (“the seeding step”). The seeding step, when used is typically carried out with particles of a choline salt of the compound of formula (I) (“the seeding particles”). Accordingly, in another embodiment, the process of the invention further comprises the step the seeding step of seeding the first admixture of Step (a) with seeding particles of a choline salt of the compound of formula (I). Prior to addition to the first admixture, the seeding particles may be combined with a suitable carrier liquid (e.g., acetone) to form a slurry which is added to the first admixture. Alternatively, the seeding particles may be added to the first admixture as dry solids, i.e., without any carrier liquid.

The size of the seeding particles, when used, can vary from about 1 μm up to about 500 μm. Thus, in one embodiment, the average diameter of the seeding particles is from about 1 μm up to about 500 μm. In another embodiment, at least about 90% of the seeding particles have a diameter of less than about 100 μm. In another embodiment, at least about 90% of the seeding particles have a diameter of less than about 50 μm. In another embodiment, at least about 90% of the seeding particles have a diameter of less than about 40 μm.

In one embodiment, the process of the invention further comprises the step of seeding the first admixture of Step (a) using seeding particles having a diameter, in one embodiment, from about 0.1 μm up to about 150 μm; in one embodiment, from about 1 μm up to about 150 μm; in another embodiment, from about 25 μm up to about 100 μm; in another embodiment, from about 0.1 μm up to about 10 μm; in another embodiment, from about 0.5 μm up to about 5 um; in another embodiment, about 75 μm; and in another embodiment, about 50 μm.

Seeding particles of a desired size can be prepared using conventional methods including, for example, milling larger particles of the choline salt of the compound of formula (I) until the desired size is obtained. The conventional milling methods include jet milling and impact milling, e.g. pin milling.

Applicants have found that morphology (e.g., size and shape) of the seeding particles influences the morphology of the choline salt of the compound of formula (I) produced by the process of the invention. For example, 90% of the choline salt particles produced by the process of the invention have a diameter of less than 100 μm when the optional seeding step is carried out using seeding particles where 90% of the seeding particles having a diameter of less than 50 μm.

The process of the invention may further comprise a polish filtration step which is used to filter the admixture of Step (a) prior to contacting with acetone in Step (b) and prior to any seeding step, when used. Accordingly, the invention relates to any of the embodiments described above for making the choline salt of the compound of formula (I), the processes further comprising the step of filtering the admixture of Step (a) prior to contacting with acetone in Step (b) and prior to any optional seeding step. The polish filtration step, when used, is typically performed at from about 25° C. up to about the refluxing temperature of the solvent; in another embodiment, from about 25° C. to about 80° C.; in another embodiment, from about 40° C. to about 70° C.; and in another embodiment, from about 65° C. to about 70° C.

In another embodiment, the process of the invention may further comprise treatment of the admixture of Step (a) with activated charcoal prior to polish filtration. Without being limited by theory, Applicants believe that treatment with activated charcoal removes trace impurities, e.g., impurities which may impart color to the final product.

The process of the invention may further comprise isolating, washing and drying the choline salt of the compound of formula (I) formed in the crystallization step. Accordingly, in one embodiment, the process of the invention described in the embodiments above further comprises the step of separating said crystalline choline salt of the compound of formula (I) of Step (c) from said admixture (“the separation step”). Any conventional method useful for solid/liquid separation may be used in the separation step including, for example, filtering, centrifuging, and/or decanting.

Once separated from the liquid phase of the admixture, the choline salt of the compound of formula (I) may be washed one or more times to remove residual impurities (“the washing step”). The amount and composition of the wash solvent(s) used in the optional washing step will vary depending on type and amount of solvent used in the admixing step. The wash solvent typically initially comprises isopropanol. It will be understood that the washing step may comprise a single wash or multiple washes with the same or different solvents. For example, after initially washing with isopropanol, the choline salt of the compound of formula (I) can be washed with an aliphatic hydrocarbon solvent that is miscible with isopropanol. Non-limiting examples of aliphatic hydrocarbons that are miscible with isopropanol useful in the washing step include butane, pentane, hexane, heptane, octane, mixtures thereof, and isomers thereof. In one embodiment, the one or more aliphatic hydrocarbons that are miscible with isopropanol are selected from hexane, heptane, octane, mixtures thereof, and isomers thereof. In another embodiment, the aliphatic hydrocarbon that is miscible with isopropanol is heptane.

The process of the invention may further comprise the step of drying the crystalline choline salt of the compound of formula (I) prepared according to any of the embodiments described above (“the drying step”). The drying step, when used, may be carried out at reduced pressure or under a dry stream of an inert gas such as nitrogen, helium, or argon. The drying step, when used, may also be carried out at temperature from about 0° C. to about 100° C.; typically, from about 50° C. to about 80° C.

In a preferred embodiment, the invention relates to a process for preparing a crystalline form of the choline salt of the compound of formula (I), the process comprising:
(a) forming a first admixture of a choline salt of the compound of formula (I) in a solvent comprising isopropanol and water;
(b) filtering said first admixture of Step (a) to provide a first filtrate;
(c) seeding said first filtrate of Step (b) with seed particles of a choline salt of the compound of formula (I) to provide a seeded filtrate, wherein at least about 90% of the seed particles of the choline salt of the compound of formula (I) have a diameter of less than about 50 μm;
(d) contacting the seeded filtrate of Step (c) with an anti-solvent comprising acetone to provide a second admixture; and
(e) allowing the choline salt of the compound of formula (I) to crystallize from the second admixture of Step (d) to provide the crystalline form of the compound of formula (I).

As noted above, Applicants have found that the process of the invention provides the choline salt of the compound of formula (I) in highly pure form. For example, the processes for preparing the choline salt of the compound of formula (I) provides product that contain less than 0.3 wt. % Compound A and Compound B based on the total weight of Compound A, Compound B, choline, and the compound of formula (I).

In one embodiment, the process of the invention provides a crystalline choline salt of the compound of formula (I) which contains, in one embodiment, less than about 0.30 wt. % of Compound A and Compound B; in another embodiment, less than about 0.20 wt. % of Compound A and Compound B; in another embodiment, less than about 0.10 wt. % of Compound A and Compound B; in another embodiment, less than about 0.05 wt. % of Compound A; in another embodiment, less than about 0.20 wt. % of Compound B; in another embodiment, less than about 0.10 wt. % of Compound B; in another embodiment, less than about 0.05 wt. % of Compound B based on the total weight of Compound A, Compound B, choline, and the compound of formula (I).

Without being limited by theory, Applicants believe that these impurities (i.e., Compounds A and B) result from the hydrolysis of the compound of formula (I) or the by-products generated in the earlier synthesis steps. Applicants believe that acetone functions as an anti-solvent and is also effective in rejecting Compound A and Compound B from the crystalline product.

In another embodiment, the invention relates to a crystalline choline salt of the compound of formula (I) which contains in one embodiment, less than about 0.30 wt. % of Compound A and Compound B; in another embodiment, less than about 0.20 wt. % of Compound A and Compound B; in another embodiment, less than about 0.10 wt. % of Compound A and Compound B; in another embodiment, less than about 0.05 wt. % of Compound A; in another embodiment, less than about 0.20 wt. % of Compound B; in another embodiment, less than about 0.10 wt. % of Compound B; in another embodiment, less than about 0.05 wt. % of Compound B based on the total weight of Compound A, Compound B, choline, and the compound of formula (I).

### Characterization

As noted above, the choline salt of the compound of formula (I) produced by the process of the invention is characterized by an X-ray powder diffraction pattern comprising 2θ angles and d-spacing values as shown in Table 1 below:

TABLE 1
2θ angles and d-spacing values for the choline salt of the compound
of formula (I) prepared according to the process of the invention.

| Angle 2θ, ° | d, Å | Relative Intensity % |
|---|---|---|
| 6.6 | 13.5 | 100.0 |
| 7.4 | 11.9 | 10.6 |
| 9.9 | 8.9 | 18.9 |
| 11.6 | 7.6 | 13.9 |
| 12.2 | 7.2 | 28.2 |
| 13.2 | 6.7 | 9.6 |
| 14.1 | 6.3 | 27.7 |
| 14.5 | 6.1 | 10.8 |
| 15.2 | 5.8 | 43.1 |
| 16.1 | 5.5 | 37.0 |
| 16.6 | 5.3 | 11.8 |
| 17.5 | 5.1 | 7.1 |
| 18.6 | 4.8 | 43.8 |
| 19.5 | 4.5 | 56.2 |
| 20.0 | 4.4 | 65.5 |
| 21.6 | 4.1 | 34.0 |
| 22.4 | 4.0 | 23.4 |
| 22.7 | 3.9 | 28.2 |
| 23.2 | 3.8 | 24.9 |
| 23.8 | 3.7 | 13.4 |
| 24.2 | 3.7 | 28.0 |
| 25.4 | 3.5 | 20.2 |
| 26.5 | 3.4 | 61.2 |
| 27.4 | 3.3 | 10.8 |
| 28.2 | 3.2 | 33.2 |
| 28.9 | 3.1 | 23.2 |
| 30.0 | 3.0 | 16.4 |
| 30.9 | 2.9 | 10.6 |
| 31.4 | 2.8 | 8.8 |
| 32.0 | 2.8 | 10.3 |
| 32.6 | 2.7 | 9.8 |
| 33.4 | 2.7 | 9.8 |
| 33.9 | 2.6 | 8.6 |
| 35.0 | 2.6 | 8.6 |
| 37.6 | 2.4 | 7.8 |
| 38.1 | 2.4 | 8.3 |

The values reported in Table 1 for the choline salt of the compound of formula (I) are substantially similar to those reported in WO 2008/156781, indicating that the processes produce substantially similar polymorphs.

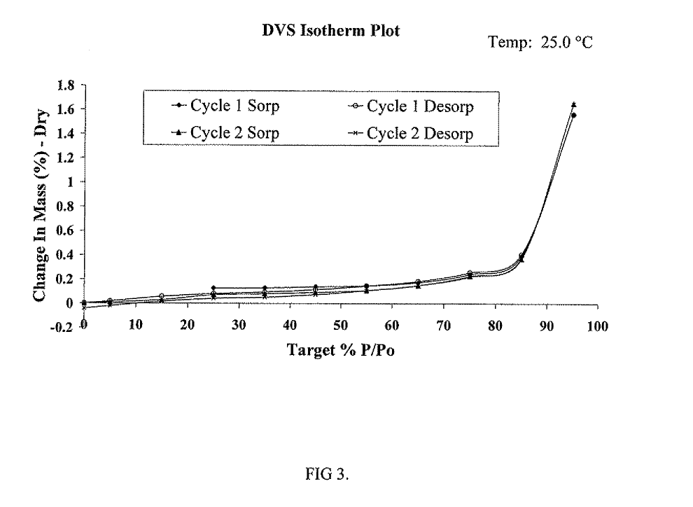

DVS data (FIG. 3) indicate that the choline salt of the invention is non-hygroscopic up to 75% relative humidity at 25° C.

### Pharmaceutical Compositions

The pharmaceutical composition of the invention may be prepared in a form suitable for inhalative, oral, intravenous, topical, subcutaneous, intramuscular, intraperitoneal, intranasal, transdermal or rectal administration.

### A) Oral Formulations

In one embodiment, the invention relates to a pharmaceutical composition of the invention that is suitable for oral administration comprising the choline salt of the invention and one or more of a pharmaceutically acceptable carrier or excipient

In another embodiment, the invention relates to a pharmaceutical composition that is suitable for oral administration consisting essentially of the choline salt of the invention.

Non-limiting examples of oral formulations include tablets, coated tablets, pills, granules or granular powder, syrups, emulsions, suspensions, or solutions, optionally together with inert and non-toxic pharmaceutically acceptable excipients or solvents

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavor enhancer, e.g., a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Capsules containing one or more active substances or combinations of active substances may for example be prepared by mixing the active substances with inert carriers such as lactose or sorbitol and packing them into gelatine capsules.

Carriers or excipients which may be used include, for example, water, pharmaceutically acceptable organic solvents such as paraffins (e.g., petroleum fractions), vegetable oils (e.g., groundnut or sesame oil), mono- or polyfunctional alcohols (e.g., ethanol or glycerol), carriers such as, e.g., natural mineral powders (e.g., kaolins, clays, talc, chalk), synthetic mineral powders (e.g., highly dispersed silicic acid and silicates), sugars (e.g., cane sugar, lactose and glucose), emulsifiers (e.g., lignin, spent sulphite liquors, methylcellulose, starch and polyvinylpyrrolidone) and lubricants (e.g., magnesium stearate, talc, stearic acid and sodium lauryl sulphate).

Tablets may additionally contain additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process.

Aqueous suspensions may be combined with various flavour enhancers or colourings in addition to the excipients mentioned above.

It will be understood that each of the oral formulations containing the choline salt of the invention may optionally contain one or more further active compounds as described below.

### B) Inhalative Formulations

In one embodiment, the invention relates to a pharmaceutical composition suitable for inhalation comprising the choline salt of the invention and one or more of a pharmaceutically acceptable carrier or excipient.

In another embodiment, the invention relates to pharmaceutical composition suitable for inhalation consisting essentially of the choline salt of the invention and at least one of a pharmaceutically carrier or excipient.

Non-limiting examples of preparations suitable for inhalation include inhalable powders, propellant-containing metered-dose aerosols and propellant-free inhalable solutions. The inhalative formulations may optionally include inert and non-toxic pharmaceutically acceptable excipients or solvents as described below.

### B.1) Powder Formulations:

The pharmaceutical composition of the invention can, in one embodiment, be in the form of an inhalable powder, optionally comprising pharmaceutically acceptable excipients.

Non-limiting examples of pharmaceutically acceptable excipients useful for powder formulations include monosaccharides (e.g., glucose or arabinose), disaccharides (e.g., lactose, saccharose, maltose, trehalose), oligo- and polysaccharides (e.g., dextran), polyalcohols (e.g., sorbitol, mannitol, xylitol), cyclodextrines (e.g., α-cyclodextrine, β-cyclodextrine, χ-cyclodextrine, methyl-β-cyclodextrine, hydroxypropyl-β-cyclodextrine), salts (e.g., sodium chloride, calcium carbonate) or mixtures of these excipients with one another. Preferably, mono- or disaccharides are used, while the use of lactose, trehalose or glucose is preferred, particularly, but not exclusively, in the form of their hydrates.

Within the scope of the inhalable powders according to the invention the excipients have in one embodiment a maximum average particle size of up to about 250 μm; in another embodiment, from about 10 to about 250 μm; in another embodiment, from about 10 to about 150 μm; and in another embodiment, from about 15 to about 80 μm.

The inhalable powders may further comprise finer excipient fractions with an average particle size of 1 to 9 μm to the excipient mentioned above. These finer excipients are also selected from the group of possible excipients listed above. In order to prepare the inhalable powders according to the invention, a micronised form of the choline salt of the invention (and the one or more further active compounds when present), preferably with an average particle size of 0.5 to 10 μm, more preferably from 1 to 6 μm, is added to the excipient mixture. Processes for producing the inhalable powders according to the invention by grinding and micronising and by finally mixing the ingredients together are known from the prior art.

In one embodiment, the invention relates to a pharmaceutical composition in the form of an inhalable powder which contains only the choline salt of the invention as its active ingredient.

The inhalable powders according to the invention may be administered using inhalers known from the prior art. Inhalable powders according to the invention which contain one or more physiologically acceptable excipients may be administered, for example, by means of inhalers which deliver a single dose from a supply using a measuring chamber as described in U.S. Pat. No. 4,570,630A, or by other means as described in DE 36 25 685 A. The inhalable powders according to the invention which contain the choline salt of the invention optionally in conjunction with a physiologically acceptable excipient may be administered, for example, using the inhaler known by the name Turbuhaler® or using inhalers as disclosed for example in EP 237507A. Preferably, the inhalable powders according to the invention which contain a physiologically acceptable excipient are packed into capsules (to produce so-called inhalettes) which are used in inhalers as described, for example, in WO 94/28958. A particularly preferred inhaler for using the inhalable powders according to the invention is the inhaler known by the name Handyhaler®.

If the inhalable powders according to the invention are packed into capsules (inhalers) for the preferred use described above, the quantities packed into each capsule should be 1 to 30 mg per capsule.

### B.2) Propellant-Containing Inhalable Aerosol

In another embodiment, the invention relates to a pharmaceutical composition in the form of a propellant-containing inhalable aerosol. Such formulations comprise the choline salt of the invention, and optionally one or more further active compounds, in dissolved and/or dispersed form.

Non-limiting examples of propellant gases useful in the propellant-containing inhalable aerosol include hydrocarbons such as n-propane, n-butane or isobutene; or halohydrocarbons such as chlorinated and/or fluorinated derivatives of methane, ethane, propane, butane, cyclopropane or cyclobutane.

In another embodiment, the propellant used in the propellant-containing inhalable aerosol is TG11 (trichlorofluoromethane), TG12 (dichlorodifluoromethane), TG134a (1,1,1,2-tetrafluoroethane), TG227 (1,1,1,2,3,3,3-heptafluoropropane), or mixtures thereof. In another embodiment, the propellant is TG134a, TG227 or a mixture thereof.

The propellant-containing inhalable aerosols according to the invention may also contain other ingredients such as co-solvents, stabilizers, surfactants, antioxidants, lubricants and pH adjusters. All these ingredients are known in the art.

The propellant-containing inhalable aerosol according to the invention may contain up to 5 wt. % of the choline salt of the invention and, optionally, one or more further active compounds. Aerosols according to the invention contain, for example, 0.002 to 5 wt. %, 0.01 to 3 wt. %, 0.015 to 2 wt. %, 0.1 to 2 wt. %, 0.5 to 2 wt. % or 0.5 to 1 wt. % of the choline salt of the invention and the optional further active compounds.

If the choline salt of the invention and optional further active compounds are present in dispersed form, the particles of active substances have, in one embodiment, an average particle size of up to about 10 μm; in another embodiment from about 0.1 to about 6 μm; and in another embodiment, from about 1 to about 5 μm.

The propellant-driven inhalation aerosols according to the invention may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterized in that they contain the propellant gas-containing aerosols described above according to the invention. The present invention also relates to cartridges fitted with a suitable valve which can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

### B.3. Propellant-Free Inhalable Aerosols

In another embodiment, the invention relates to a pharmaceutical composition in the form of a propellant-free inhalable aerosol.

The propellant-free inhalable aerosol of the invention is in the form of a solution or suspension. Propellant-free inhalable solutions and suspensions according to the invention contain, for example, aqueous or alcoholic, preferably ethanolic solvents, optionally ethanolic solvents mixed with aqueous solvents. If aqueous/ethanolic solvent mixtures are used the relative proportion of ethanol compared with water is not limited but preferably the maximum is up to 70 percent by volume, more particularly up to 60 percent by volume of ethanol. The remainder of the volume is made up of water. The solutions or suspensions containing the choline salt of the invention and optional further active compound, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g., as flavorings, antioxidants or complexing agents, such as citric acid or ascorbic acid, for example. According to the invention, it is particularly preferred to use hydrochloric acid to adjust the pH.

According to the invention, the addition of editic acid (EDTA) or one of the known salts thereof, sodium editate, as stabilizer or complexing agent is unnecessary in the present formulation. Other embodiments may contain this compound or these compounds. In a preferred embodiment the content based on sodium editate is less than 100 mg/100 ml, preferably less than 50 mg/100 ml, more preferably less than 20 mg/100 ml. Generally, inhalable solutions in which the content of sodium editate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g., alcohols, particularly isopropyl alcohol, glycols, particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavorings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

In one embodiment, the propellant-free inhalable solution comprises water, the choline salt of the invention, and a preservative. In another embodiment, the propellant-free inhalable solution comprises water, the choline salt of the invention, and a preservative selected from benzalkonium chloride and sodium editate. In yet another embodiment, the propellant-free inhalable solution comprises water, the choline salt of the invention, and benzalkonium chloride. In yet another embodiment, the propellant-free inhalable solution comprises water, the choline salt of the invention, and a preservative which is not sodium editate.

The propellant-free inhalable solutions according to the invention can be administered using inhalers of the kind which are capable of nebulizing a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 20 and 30 μL of active substance solution can be nebulized in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulizers (devices) described therein are known by the name Respimat®.

In one embodiment, the invention relate to a pharmaceutical composition in the form of an inhalable solution optionally containing other co-solvents and/or excipients.

In another embodiment, the invention relates to a pharmaceutical composition in the form of an inhalable solution comprising at least one co-solvent containing hydroxyl groups or other polar groups, e.g., alcohols, particularly isopropyl alcohol glycols, particularly propyleneglycol, polyethyleneglycol, polypropyleneglycol, glycolether, glycerol, polyoxyethylene alcohols; and polyoxyethylene fatty acid esters.

In yet another embodiment, the invention relates to pharmaceutical composition in the form of an inhalable solution containing excipients selected from surfactants, stabilisers, complexing agents, antioxidants and/or preservatives, flavourings, pharmacologically acceptable salts and/or vitamins.

When the propellant-free inhalable aerosols comprise a further active compound, the doses applicable for the combinations according to the invention are to be understood as referring to doses per single application. However, it will be understood that these do not exclude the possibility of administering the combinations according to the invention multiple times. Depending on the medical need patients may receive also multiple inhalative applications. For example, patients may receive the combinations according to the invention for instance two or three times (e.g., two or three puffs with a powder inhaler, an MDI etc.) in the morning of each treatment day. As the aforementioned dose examples are only to be understood as dose examples per single application (i.e., per puff) multiple application of the combinations according to the invention leads to multiple doses of the aforementioned examples. The application of the compositions according to the invention can be for instance once a day, or depending on the duration of action of the agents twice a day, or once every 2 or 3 days.

It will be understood that the aforementioned dosages are to be understood as examples of metered doses only, i.e., the aforementioned doses are not to be understood as the effective doses of the combinations according to the invention that do in fact reach the lung. It is clear for the person of ordinary skill in the art that the delivered dose to the lung is generally lower than the metered dose of the administered active ingredients.

### The Unit Dose Form and Methods of Administration

As noted above, the pharmaceutical composition of the invention may be administered in the form of a preparation suitable for inhalative, oral, intravenous, topical, subcutaneous, intramuscular, intraperitoneal, intranasal, transdermal or rectal administration. The pharmaceutical composition of the invention is applied to the patient as a unit dose form.

As used herein, the phrase “unit dose form” refers to the actual product, through which the pharmaceutical composition of the invention is administered to the patient. Non-limiting examples of unit dose forms include tablets, lozenges, capsules, inhalation powder capsules, unit dose vials, metered doses provided by a metered dose inhaler (MDI), injection vials and others commonly known by the skilled artisan.

In one embodiment, the invention relates to a method of orally administering the pharmaceutical composition to a patient in need thereof. Oral administration can be done one or more times per day in order to achieve the daily dosage for the patient. In another embodiment, the choline salt of the invention is administered orally twice a day. In another embodiment, the choline salt of the invention is administered orally once a day.

In another embodiment, the invention relates to an inhalative method for administering the pharmaceutical composition to a patient in need thereof. In yet another embodiment, the inhalative method comprises a pharmaceutical compositions selected from inhalable powders, propellant-containing metered-dose aerosols and propellant-free inhalable solutions. In another embodiment, the inhalative the inhalative method comprises an inhalable powder. In another embodiment, the inhalative the inhalative method comprises a propellant-containing metered-dose aerosol. And in another embodiment, the inhalative the inhalative method comprises a propellant-free inhalable solution.

In another embodiment, the invention relates to the use of a suppository to administer the pharmaceutical composition to a patient in need thereof. Suitable suppositories may be made for example by mixing with carriers provided for this purpose, such as neutral fats or polyethyleneglycol or the derivatives thereof.

The pharmaceutical composition of the invention can be applied to the patient via the unit dose form in one administration or in more than one sub-administration. In one embodiment, the daily dosages mentioned herein above are administered to the patient in a three-times-daily (t-d) administration scheme; in another embodiment, the daily dosages mentioned herein above are administered to the patient in a twice-daily (b-i-d) administration scheme; and in another embodiment, the daily dosages mentioned herein above are administered to the patient in a once-daily (q-d) administration scheme.

In one embodiment, the unit dose form comprises the choline salt of the invention in an amount of from about 1 mg to about 1000 mg; in another embodiment, from about 5 mg to about 800 mg; in another embodiment, from about 10 mg to about 700 mg; in another embodiment, from about 15 mg to about 600 mg; in another embodiment, from about 20 mg to about 500 mg; and in another embodiment, from about 25 mg to about 400 mg.

### Medical Indications

The choline salt of the invention shows excellent CRTH2 antagonistic activity. It is, therefore, suitable for the prophylaxis and treatment of diseases associated with CRTH2 activity. It has been found that the pharmaceutical compositions described herein have a beneficial effect in terms of bronchospasmolysis and reduction of inflammations in the airways; allergic diseases of the oro-naso pharynx, skin or the eyes; inflammatory diseases of the joints; and inflammatory bowel disease.

In one embodiment, the invention relates to the treatment of an indication (A) selected from:
diseases of the airways and lungs which are accompanied by increased or altered production of mucus and/or inflammatory and/or obstructive diseases of the airways such as acute bronchitis, chronic bronchitis, chronic obstructive bronchitis (COPD), cough, pulmonary emphysema;
allergic or non-allergic rhinitis or sinusitis, chronic sinusitis or rhinitis;
nasal polyposis, chronic rhinosinusitis, acute rhinosinusitis;
asthma, allergic bronchitis, alveolitis, Farmer's disease, hyper-reactive airways;
bronchitis or pneumonitis caused by infection, e.g., by bacteria or viruses or helminthes or fungi or protozoons or other pathogens;
pediatric asthma, bronchiectasis;
pulmonary fibrosis;
adult respiratory distress syndrome, bronchial and pulmonary edema;
bronchitis or pneumonitis or interstitial pneumonitis caused by different origins e.g., aspiration, inhalation of toxic gases, vapors;
bronchitis or pneumonitis or interstitial pneumonitis caused by heart failure, X-rays, radiation, chemotherapy;
bronchitis or pneumonitis or interstitial pneumonitis associated with collagenosis, e.g., lupus erythematodes, systemic scleroderma;
lung fibrosis, idiopathic pulmonary lung fibrosis (IPF), interstitial lung diseases or interstitial pneumonitis of different origin, including asbestosis, silicosis, M. Boeck or sarcoidosis, granulomatosis;
cystic fibrosis or mucoviscidosis; or
α-1-antitrypsin deficiency.

Thus, in one embodiment, the invention relates to the use of a pharmaceutical composition of the invention for the manufacture of a medicament for treating respiratory diseases and conditions selected from indications (A) described above.

In another embodiment, the invention relates to a method of treating an indication selected from (A) above comprising administering a therapeutically effective amount of pharmaceutical composition of the invention to a patient in need thereof.

In yet another embodiment, the invention relates to a method of treating an indication (A) selected from chronic bronchitis, chronic obstructive bronchitis (COPD), chronic sinusitis, nasal polyposis, allergic rhinitis, chronic rhinosinusitis, acute rhinosinusitis, and asthma, the method comprising administering a therapeutically effective amount of pharmaceutical composition of the invention to a patient in need thereof.

In one embodiment, the invention relates to the treatment of an indication (B) selected from:
inflammatory diseases of the gastrointestinal tract of various origins such as inflammatory pseudopolyps, Crohn's disease, ulcerative colitis;
inflammatory diseases of the joints, such as rheumatoid arthritis; or
allergic inflammatory diseases of the oro-nasopharynx, skin or the eyes.

Thus, in one embodiment, the invention relates to the use of a pharmaceutical composition of the invention for the manufacture of a medicament for treating respiratory diseases and conditions selected from indications (B) described above.

In another embodiment, the invention relates to a method of treating an indication selected from indications (B) comprising administering a therapeutically effective amount of a pharmaceutical composition of the invention to a patient in need thereof.

In another embodiment, the invention relates to a method of treating an indication (B) selected from allergic inflammatory diseases of the oro-nasopharynx, skin or the eyes, Crohn's disease or ulcerative colitis.

In one embodiment, the present invention relates to a method for making a medicament for treating any of the aforementioned diseases and conditions by using a pharmaceutical composition of the invention, optionally containing one or more one further active compounds.

In another embodiment, the present invention relates to a method for making a medicament for treating asthma and allergic and non-allergic rhinitis by using a pharmaceutical composition comprising the choline salt of the invention, and optionally containing one or more further active compounds.

### Further Active Compounds

The pharmaceutical compositions of the invention can optionally comprise one or more additional active compound. Accordingly, in one embodiment, the invention relates to a pharmaceutical composition comprising a therapeutically effective amount of the choline salt of the invention, at least one of a pharmaceutically acceptable carrier or excipient, and at least one of a further active compound (“the combinations”). In another embedment, the invention relates to a method of administering the choline salt of the invention and at the least one of a further active compound to a patient in need thereof.

The actives of the combinations may be administered simultaneously, separately or sequentially. The preferred route of administration depends on the indication to be treated.

In one embodiment, the at least one further active compound is selected from the classes consisting of β2-adrenoceptor-agonists (short and long-acting beta mimetics), anti-cholinergics (short and long-acting), anti-inflammatory steroids (oral and topical corticosteroids), dissociated-glucocorticoidmimetics, PDE3 inhibitors, PDE4-inhibitors, PDE7-inhibitors, LTD4 antagonists, EGFR-inhibitors, PAF antagonists, Lipoxin A4 derivatives, FPRL1 modulators, LTB4-receptor (BLT1, BLT2) antagonists, Histamine receptor antagonists, PI3-kinase inhibitors, inhibitors of non-receptor tyrosine kinases as for example LYN, LCK, SYK, ZAP-70, FYN, BTK or ITK, inhibitors of MAP kinases as for example p38, ERK1, ERK2, JNK1, JNK2, JNK3 or SAP, inhibitors of the NF-κB signalling pathway as for example IKK2 kinase inhibitors, iNOS inhibitors, MRP4 inhibitors, leukotriene biosynthese inhibitors as for example 5-Lipoxygenase (5-LO) inhibitors, cPLA2 inhibitors, Leukotriene A4 Hydrolase inhibitors or FLAP inhibitors, Non-steroidal anti-inflammatory agents (NSAIDs), DP1-receptor modulators, Thromboxane receptor antagonists, CCR1 antagonists, CCR2 antagonists, CCR3 antagonists, CCR4 antagonists, CCR5 antagonists, CCR6 antagonists, CCR7 antagonists, CCR8 antagonists, CCR9 antagonists, CCR10 antagonists, CXCR1 antagonists, CXCR2 antagonists, CXCR3 antagonists, CXCR4 antagonists, CXCR5 antagonists, CXCR6 antagonists, CX3CR1 antagonists, Neurokinin (NK1, NK2) antagonists, Sphingosine 1-Phosphate receptor modulators, Sphingosine 1 phosphate lyase inhibitors, Adenosine receptor modulators as for example A2a-agonists, modulators of purinergic receptors as for example P2X7 inhibitors, Histone Deacetylase (HDAC) activators, Bradykinin (BK1, BK2) antagonists, TACE inhibitors, PPAR gamma modulators, Rho-kinase inhibitors, interleukin 1-beta converting enzyme (ICE) inhibitors, Toll-Like receptor (TLR) modulators, HMG-CoA reductase inhibitors, VLA-4 antagonists, ICAM-1 inhibitors, SHIP agonists, GABAa receptor antagonist, ENaC-inhibitors, Melanocortin receptor (MC1R, MC2R, MC3R, MC4R, MC5R) modulators, CGRP antagonists, Endothelin antagonists, mucoregulators, immunotherapeutic agents, compounds against swelling of the airways, compounds against cough, CB2 agonists, retinoids, immunosuppressants, mast cell stabilizers, methylxanthine, opioid receptor agonists, laxatives, anti-foaming agents, antispasmodic agents, 5-HT4 agonists, and any combination thereof.

In another embodiment, the at least one further active compound is a PDE4 inhibitor. In yet another embodiment, the at least one further active compound is the PDE4 inhibitor Roflumilast.

In another embodiment, the at least one further active compound is a LTD4 antagonist. In yet another embodiment, the at least one further active compound is a LTD4 antagonist selected from montelukast, pranlukast and zafirlukast.

In another embodiment, the at least one further active compound is a histamine receptor antagonist. In yet another embodiment the at least one further active compound is a histamine receptor antagonist selected from azelastine, cetirizine, desloratidine, ebastine, epinastine, fexofenadine, hydroxyzine, ketotifen, levocetirizine, loratadine and olopatadine.

In another embodiment, the at least one further active compound is a 5-LO inhibitor. In yet another embodiment the at least one further active compound is the 5-LO inhibitor Zileuton.

In another embodiment, the at least one further active compound is a CCR5 antagonist. In yet another embodiment the at least one further active compound is the CCR5 antagonist Maraviroc.

In another embodiment, the at least one further active compound is a CCR9 antagonist. In yet another embodiment the at least one further active compound is the CCR9 antagonist Trafficet.

In another embodiment, the at least one further active compound is a Sulfonamide. In yet another embodiment the at least one further active compound is a Sulfonamide selected from Mesalazine and Sulfasalazine.

The choline salt of the invention and at least one of a further active compound may be combined in a single preparation, e.g., as a fixed dose combination comprising the active agents in one formulation together, or contained in two or more separate formulations, e.g., as a kit of parts adapted for simultaneous, separate or sequential administration. When the pharmaceutical compositions of the invention comprise one or more further active compounds, a single preparation is preferred.

When used in combination with a further active compound, the inhalable powders combination according to the invention may be prepared and administered either in the form of a single powder mixture which contains both the choline salt of the invention and the one or more further active compounds, or in the form of separate inhalable powders which comprise only the choline salt of the invention or the one or more further active compounds.

The daily dosage of the at least one further active compound, when present, is from about 1 mg to about 1000 mg; in another embodiment, from about 2 mg to 800 mg; in another embodiment, from about 3 mg to about 500 mg: in another embodiment, from about 4 mg to about 300 mg; in another embodiment, from about 5 mg to about 200 mg; and in another embodiment, from about 6 mg to about 150 mg.

### Experimental

The choline salt of the compound of formula (I) was characterized using X-ray powder diffraction (XRPD), differential scanning calorimetry (DSC), thermogravimetric analysis (TGA), vapor sorption/desorption, and elemental analysis.

XRPD data were recorded with a Rigaku Miniflex II powder diffractometer (The Woodlands, Tex.). The radiation was CuKa (30 kV, 15 mA). Data were collected at 25° C. from 3 to 35 degrees 2θ at 0.02 degrees per step and 1.67 sec per step. Samples were prepared on Silicon (510) specimen holders as a thin layer of powdered material without solvent.

DSC was carried out using TA Instruments Q1000 Differential Scanning calorimeter. Samples were placed in sealed aluminum pans for analysis with an empty aluminum pan as the reference. A heating rate of 10° C./min was employed over a temperature range from 20° C. to 300° C.

TGA was carried out using TA Instruments Q500 Thermogravimetric analyzer. Samples were placed into an platinum sample pan. A heating rate of 10° C./min was employed over a temperature range from 25° C. to 300° C.

Vapor sorption/desorption was carried out using Surface Measurement Systems DVS-HT. Samples were placed into a foil insert placed on a sample pan. Water sorption and desorption of the sample was observed at 25° C. with stepwise change of relative humidity from 5% to 95% with two cycles of sorption/desorption. The equilibrium point of each step was reached when 0.002% of weight change was reached.

Determination of particle size distribution was carried out using a Sympatec HELOS System H1588 with a RODOS/M Dry Powder Dispersion System. Particle size distribution (PSD) results are shown in Table 2.

HPLC analysis was carried out using an Agilent 1200 chromatographic system. Separation was achieved on a Halo C18 normal phase column (4.6×150 mm, 2.7 μm). The mobile phases were 0.1% H₃PO₄, 20 nM NH4 PF6 in water and acetonitrile. The diluent was methanol. The flow rate was 1.4 mL/min and injection volume was 5 μL. The UV detection wavelength used for quantification was 254 nm. The limit of detection was 0.05% based on area.

### Preparation of Seed Crystals:

Seed crystals of the choline salt of the compound of formula (I) are prepared by milling the compound of formula (I). The milling is carried out using a jet mill where the particle size reduction is accomplished by the particle-particle impact induced by air-jet, or an impact mill where the particle size reduction is accomplished by impact of particles with the moving parts or walls of the mill. The milling is continued until 90% of the particles have a diameter of less than 50 mm. The milled particles are then collected and stored at ambient condition until used.

### Example 1a

Step 1. A seed slurry is prepared as described above by jet milling (Fluid Energy Loop Mill) the choline salt of formula (I). The milled solids (0.2 g) are then suspended in 21.25 g acetone prior to use.

Step 2. A suspension of the base-free form of [4,6-bis(dimethylamino)-2-(4-{[4-(trifluoromethyl)benzoyl]amino}benzyl)pyrimidin-5-yl]acetic acid (20 g, 39.88 mmol) and isopropanol (30 g) are heated to 60° C. and treated with 11.31 g of a solution of 44.86 wt. % aqueous solution of choline hydroxide (5.07 g, 41.86 mmol). 2.718 g of H₂O is used to chase the choline hydroxide bottle. The resultant solution is held at 60° C. for 0.5-1 hour and filtered. A solution of isopropanol (1.5 g) and H₂O (0.448 g) solution is used to chase the reactor and filter. The resultant combined filtrate is then cooled to 40° C., seeded with the seed slurry, and stirred at 40° C. for 30 minutes. The resultant suspension is then cooled over the course of 1.5 hours to 0° C. with simultaneous slow addition of acetone (191.25 g). The suspension is maintained at 0° C. for 4 hours and filtered. The resultant solids are washed 2×25 mL with isopropanol and 1×20 mL with heptane. The solids are then dried under reduced pressure at 70° C. to provide the choline salt of [4,6-bis(dimethylamino)-2-(4-{[4-(trifluoromethyl)benzoyl]amino}benzyl)pyrimidin-5-yl]acetic acid as an off-white to white crystalline solid. Yield: 22.4 g, 37 mmol, 92%. No impurities (including Compound A and Compound B are detected using HPLC (limit of detection 0.05% by area). The results indicate the product is essentially free of any impurities.

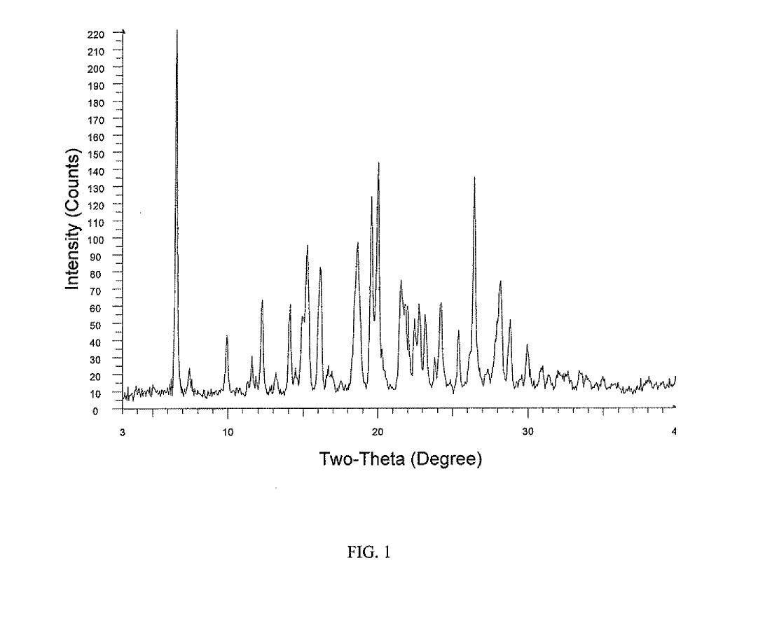

The product has an X-ray powder diffraction pattern as depicted in FIG. 1 and Table 1 which are substantially similar to those reported for the choline salt described in WO 2008/156781.

Microscopic examination of the product show small prismatic crystals. PSD data are shown in Table 2.

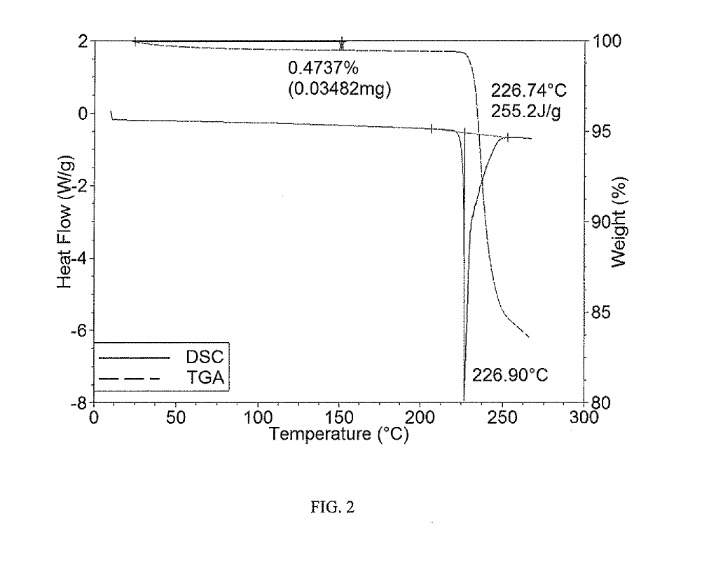

Thermal analyses of the product are shown in FIG. 2 (DSC and TGA) and FIG. 3 (DVS).

### Example 1b

Step 1. Seeding crystals are prepared as described above by impact milling (opposed jet mill with dynamic classifier) the choline salt of formula (I). The milled solids are not suspended prior use but added directly.

Step 2. The free-acid form of the compound of formula (I) (50 g, 0.100 mol) is suspended in 2-propanol (85 mL) and water (4.5 mL) at 25° C. The resultant colorless suspension is warmed to 70° C. and treated with 28.2 g of a 45% aqueous solution of choline hydroxide (12.6 g; 0.105 mmol). The resultant yellow solution is filtered, and the filter washed with 2-propanol (82 mL). The combined filtrates are cooled to 40° C. and seeded with 0.5 g of the choline salt of the compound of formula (I). The resultant suspension is stirred for about 30 minute then cooled to about 5° C. within 90 minutes with the simultaneous slow addition of acetone (300 mL). The mixture is then filtered. The collected solids are washed with 2-propanol (125 mL) and dried at 60° C. under reduced pressure for about 12 hours to provide the choline salt of [4,6-bis(dimethylamino)-2-(4-{[4-(trifluoromethyl)benzoyl]amino}benzyl)pyrimidin-5-yl]acetic acid. Yield: 53.4 g; 88.2 mmol, 88%. The purity of the compounds is >99.95% using HPLC.

### Comparative Example 2

A suspension of the base-free form of [4,6-bis(dimethylamino)-2-(4-{[4-(trifluoromethyl)benzoyl]amino}benzyl)pyrimidin-5-yl]acetic acid (60 g, 119.6 mmol) and ethanol (347.04 g) containing 2.5 wt. % toluene is heated to 65° C. and treated with 33.82 g of a solution of 45 wt. % methanolic solution of choline hydroxide (15.21 g, 125.6 mmol). The resultant solution is held at 65° C. for 0.5 hour and filtered. The resultant filtrate is then cooled to 50° C., seeded with the 0.3 g of the dry seed crystals of the compound of formula (I) as described above, and stirred at 50° C. for 30 minutes. The resultant suspension is then cooled over the course of 1 hour to 0° C. with simultaneous slow addition of heptane (347.04 g). The resultant suspension is maintained at 0° C. for 3 hours and filtered. The resultant solids are washed 1×120 g with heptane. The solids are then dried under reduced pressure at 70° C. to provide the choline salt of [4,6-bis(dimethylamino)-2-(4-{[4-(trifluoromethyl)benzoyl]amino}benzyl)pyrimidin-5-yl]acetic acid as an off-white to white crystalline solid. Yield: 64 g, 105.8 mmol, 88.9%. Purity: 99.2% based on HPLC. HPLC analysis further indicates the product contains 0.09% by area of Compound A and 0.26% by area of Compound B.

The product has an X-ray powder diffraction pattern as depicted in Table 1 and substantially similar to that reported for the choline salt described in WO 2008/156781.

Microscopic examination of the product show large rhombhedral crystals. PSD data are shown in Table 2.

### Comparative Example 3

The choline salt of the compound of formula (I) is prepared in a manner similar to that described in Example 5 of WO2008/156781. A suspension of the base-free form of [4,6-bis(dimethylamino)-2-(4-{[4-(trifluoromethyl)benzoyl]amino}benzyl)pyrimidin-5-yl]acetic acid (3.493 g, 6.96 mmol), isopropanol (50 ml) and 1.943 g of a solution of 50 wt. % aqueous solution of choline hydroxide (0.97 g, 8 mmol) is heated to reflux until a clear solution is obtained. The resultant solution is allowed to cool to 25° C., and stirred for an additional 2 hours. The resultant mixture is filtered, and the solids washed with 28 mL of isopropanol/heptane (1:1, v/v). The solids are then dried under reduced pressure at 25° C. to provide the product as yellow, large plate-like solids. Yield: 3.49 g, 5.7 mmol, 82%. Purity: 99.2% by area based on HPLC. HPLC analysis further indicates the product contains 0.10% of Compound A and 0.22% of Compound B.

PSD data are shown in Table 2.

### Comparative Example 4

The choline salt of the compound of formula (I) is prepared in a manner similar to that described in Example 1 above except that no acetone is added to the reaction mixture when the mixture is cooled from 40° C. to 0° C. Yield: 31%. Purity: 99.3% by area based on HPLC. HPLC analysis further indicates the product contains 0.10% of Compound A and 0.26% of Compound B.

PSD data are shown in Table 2.

TABLE 2
Particle size distribution (PSD) for the compounds prepared
according to Example 1 and Comparative Examples 2-4.

| Preparation | | | |
|---|---|---|---|
| Method | % <10 μM | % <50 μM | % <90 μM |
| Ex. 1 | 5.13 | 16.67 | 35.45 |
| Comp. Ex. 2 | 11.57 | 60.13 | 119.62 |
| Comp. Ex. 3 | 6.87 | 57.62 | 181.03 |
| Comp. Ex. 4* | 3.01 | 13.62 | 37.98 |

*Low yield (31%)

The results shown above in Examples 1a and 2a and Comparative Examples 2 to 4 indicate that the process of the invention produces a choline salt of the compound of formula (I) having high purity and the desired, small particle size useful for a component of a pharmaceutical composition without the need for milling.

The examples set forth above are provided to give those of ordinary skill in the art with a complete disclosure and description of how to make and use the embodiments, and are not intended to limit the scope of the disclosure. Modifications of the above-described modes for carrying out the disclosure that are obvious to persons of skill in the art are intended to be within the scope of the inventions. All publications, patents and patent applications cited in this specification are incorporated herein by reference as if each such publication, patent or patent application were specifically and individually indicated to be incorporated herein by reference.

What is claimed is:

1. A process for preparing a crystalline form of the choline salt of the compound of formula (I), 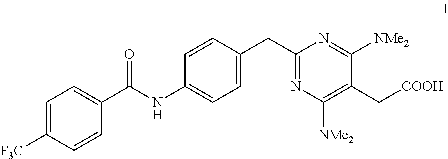
the process comprising:
(a) forming a first admixture of a choline salt of the compound of formula (I) in a solvent comprising isopropanol and water;
(b) contacting the first admixture of Step (a) with an anti-solvent to provide a second admixture; and
(c) allowing the choline salt of the compound of formula (I) to crystallize from said second admixture of Step (b) to provide the crystalline form of the compound of formula (I).

2. The process of claim 1, wherein the first admixture of Step (a) is prepared by combining the free acid form of the compound of formula (I) with choline hydroxide.

3. The process of claim 1, wherein Step (a) is carried out with a solvent consisting essentially of isopropanol and water.

4. The process of claim 1, wherein the first admixture formed in Step (a) is polish filtered prior to carrying out Step (b).

5. The process of claim 5, wherein the first admixture formed in Step (a) is treated with activated charcoal prior to polish filtration.

6. The process of claim 1, wherein the first admixture formed in Step (a) is seeded with seeding particles prior to carrying out Step (b).

7. The process of claim 6, wherein the seeding particles have a diameter of from about 0.1 μm up to about 150 μm.

8. The process of claim 7, wherein the seeding particles have a diameter of from about 25 μm up to about 100 μm.

9. The process of claim 7, wherein the seeding particles have a diameter of from about 0.5 μm up to about 5 um.

10. The process of claim 1, wherein the anti-solvent used in Step (c) is selected from acetone, isopropanol, and heptane.

11. The process of claim 1, wherein the anti-solvent used in Step (c) is acetone.

12. A crystalline choline salt of [4,6-bis(dimethylamino)-2-(4-{[4-(trifluoromethyl)benzoyl]amino}benzyl)pyrimidin-5-yl]acetic acid, wherein said crystalline choline salt contains less than about 0.30 wt. % of 2-(4-(dimethylamino)-6-hydroxy-2-4-(trifluoromethyl)benzamido)pyrimidine-5-yl)acetic acid (Compound A) and N-(4-((5-(cyanomethyl)-4,6-bis(dimethylamino)pyrimidin-2-yl)methyl)phenyl)-4-(trifluoromethyl)benzamide (Compound B) based on the total weight of Compound A, Compound B, and [4,6-bis(dimethylamino)-2-(4-{[4-(trifluoromethyl)benzoyl]amino}benzyl)pyrimidin-5-yl]acetic acid.

13. The crystalline choline salt of claim 12 wherein said crystalline choline salt contains less than about 0.10 wt. % of Compound A and Compound B based on the total weight of Compound A, Compound B, and [4,6-bis(dimethylamino)-2-(4-{[4-(trifluoromethyl)benzoyl]amino}benzyl)pyrimidin-5-yl]acetic acid.

14. A pharmaceutical composition comprising the crystalline choline salt of [4,6-bis(dimethylamino)-2-(4-{[4-(trifluoromethyl)benzoyl]amino}benzyl)pyrimidin-5-yl]acetic acid of claim 12 and at least one of a pharmaceutically acceptable carrier or excipient.

15. A method of treating a disease associated with CRTH2 activity, the method comprising administering a therapeutically effective amount of pharmaceutical composition comprising the compound of claim 12 to a patient in need thereof.
